(19) United States
(12) Patent Application Publication
(10) Pub. No.: US 2009/0326646 A1
(43) Pub. Date: Dec. 31, 2009

(54) Methods and Apparatus for Multiple Cured Formulation Coated Stents

(75) Inventors: Ronan Burke, Lavallyconor (IE); Conor Shanaghy, Athlone (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/147,857

(22) Filed: Jun. 27, 2008

(51) Int. Cl. A61F 2/06

(52) U.S. Cl. 623/1.46

(57) ABSTRACT

The methods and apparatus of the present disclosure in a broad aspect provide stents with multiple cured formulations. Selective curing of formulations on a stent framework, such as by ultraviolet light, results in stents having multiple cured formulations as coatings which may or may not be layered in uniform or non-uniform fashion.

### FIELD OF THE INVENTION

The present disclosure relates to methods and apparatus for multiple cured formulation coated stents useful for treating, for example, vascular diseases and conditions.

### BACKGROUND OF THE INVENTION

Stents are generally cylindrical shaped devices that are radially expandable to hold open a segment of a vessel or other anatomical lumen after implantation into the body lumen. Stents have been developed with coatings to deliver drugs or other therapeutic agents. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the length of the stent to contract and the diameter to expand. For a self-expanding stent, commonly a sheath is retracted, allowing expansion of the stent.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion, thereby increasing the inner diameter of the affected vessel. The increased interior vessel diameter facilitates improved blood flow. Soon after the procedure, however, a significant proportion of treated vessels re-narrow.

Restenosis associated with interventional procedures such as balloon angioplasty may occur by two mechanisms: thrombosis and intimal hyperplasia. During angioplasty, a balloon is inflated within an affected vessel thereby compressing the blockage and imparting a significant force, and subsequent trauma, upon the vessel wall. The natural antithrombogenic lining of the vessel lumen may become damaged thereby exposing thrombogenic cellular components, such as matrix proteins. The cellular components, along with the generally antithrombogenic nature of any implanted materials (e.g., a stent), may lead to the formation of a thrombus, or blood clot. The risk of thrombosis is generally greatest immediately after the angioplasty.

The second mechanism of restenosis is intimal hyperplasia, or excessive tissue re-growth. The trauma imparted upon the vessel wall from the angioplasty is generally believed to be an important factor contributing to hyperplasia. This exuberant cellular growth may lead to vessel “scarring” and significant restenosis. The risk of hyperplasia associated restenosis is usually greatest 3 to 6 months after the procedure.

Prosthetic devices, such as stents or grafts, may be implanted during interventional procedures such as balloon angioplasty to reduce the incidence of vessel restenosis. To improve device effectiveness, stents may be coated with one or more therapeutic agents providing a mode of localized drug delivery. The therapeutic agents are typically intended to limit or prevent the aforementioned mechanisms of restenosis. For example, antithrombogenic agents such as heparin or clotting cascade IIb/IIIa inhibitors (e.g., abciximab and eptifibatide) may be coated on the stent thereby diminishing thrombus formation. Such agents may effectively limit clot formation at or near the implanted device. Some antithrombogenic agents, however, may not be effective against intimal hyperplasia. Therefore, the stent may also be coated with antiproliferative agents or other compounds to reduce excessive endothelial re-growth. Therapeutic agents provided as coatings on implantable medical devices may effectively limit restenosis and reduce the need for repeated treatments.

Stents can be coated with a polymer or combination of a polymer and a pharmaceutical agent or drug. In many of the current medical devices or stent coating methods, a composition of a drug and a polymer in a solvent is applied to a device to form a substantially uniform layer of drug and polymer. A common solvent for the polymers and drugs employed is usually required, and techniques have been developed to micronize the drugs into small particles so that the drugs can be suspended in the polymer solution.

The above discussed methods of coating may produce stents with drug and/or polymer formulations. These methods may even produce coatings that contain different formulations. However, these produced coatings having formulations would generally be uniform relative to each other and typically would be on top of each other in a uniform fashion. However, it may be desirable to have stents with coatings which are not uniform relative to each other, especially those coatings which are not on top of each other. This is because when multiple coatings with multiple formulations are on top of each other they will elute one at a time and not simultaneously. When different formulations such as those containing different antirestenosis drugs need to elute at the same time, it would be desirable to have stents with coatings that are not uniform relative to each other, especially those having coatings which are not on top of each other.

Therefore, there is a significant need for apparatus and associated methods of coating stents which allow multiple formulations containing drugs and/or polymers that are not necessarily on top of each other.

### SUMMARY OF THE INVENTION

These and are other objects are achieved by the methods and apparatus of the present disclosure, which, in a broad aspect, provide stents coated with multiple cured formulations. This is achieved by selectively curing formulations which may contain drugs and/or polymers after their application onto a stent framework. The coated stents produced by the present methods are capable of eluting each of the different drug and/or polymer formulations at the same time, with the same or different rates. They may also elute at different times. The multiple formulations are not necessarily on top of each other and do not have to elute one after the other.

In a broad aspect, the present methods include the steps of providing a stent framework; putting the stent framework on a fixture; applying a curable formulation onto the stent to provide a curably coated stent; covering the curably coated stent with a glass mask etched with a pattern which allows selective exposure of the curable formulation; curing the curable formulation on the covered stent thereby forming a substantially cured coated stent; removing the glass mask; applying a development solution to the substantially cured coated stent to remove uncured formulation; drying off excess development solution; and repeating the coating and curing steps with a glass mask etched with a different pattern and a different curable formulation.

In another embodiment, the curable formulation is a photosensitive curable formulation comprising a photoactive compound. This photosensitive formulation can be sensitive to ultraviolet (UV) light. The curing of the curable formulation can be performed with ultraviolet light. The curable formulation may further comprise a bioactive agent and/or a polymer. The bioactive agent can be but not limited to antirestenotic drug or be selected from the group consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof; and the polymer can be selected from the group consisting of polyurethane, silicone, polyolefin, polyisobutylene, ethylene-alphaolefin copolymer, acrylic polymer and copolymer, ethylene-co-vinylacetate, polybutylmethacrylate, vinyl halide polymer and copolymer, polyvinyl chloride, polyvinyl ether, polyvinyl methyl ether, polyvinylidene halide, polyvinylidene fluoride, polyvinylidene chloride, polyacrylonitrile, polyvinyl ketone, polyvinyl aromatic, polystyrene, polyvinyl ester, polyvinyl acetate, ethylene-methyl methacrylate copolymer, acrylonitrile-styrene copolymer, ABS resin, ethylene-vinyl acetate copolymer, polyamide, alkyd resin, polycarbonate, polyoxymethylene, polyimide, polyether, epoxy resin, polyurethane, rayon, rayon-triacetate, cellulose, cellulose acetate, cellulose butyrate, cellulose acetate butyrate; cellophane, cellulose nitrate, cellulose propionate, cellulose ether, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylate, poly(trimethylene carbonate), poly(iminocarbonate), copoly(ether-esters), polyalkylene oxalate, polyphosphazene, fibrin, fibrinogen, cellulose, starch, collagen and hyaluronic acid, and combinations thereof.

The method steps can be repeated once to have two different formulations as coatings. The method steps can be repeated twice to have three different formulations as coatings; and repeated three times to have four different formulations as coatings and so on and so forth. The stent framework provided for coating may comprise a metallic base. The metallic base may be made of material selected from the group consisting of stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof. The stent can also be coated with a preparatory base coat such as a primer before any application of a curable formulation. The primer may be selected from the group consisting of parylene, polyurethane, epoxy, polyimide, polysulfone, and pellathane. Alternatively, the stent framework may comprise a polymeric base.

Another embodiment of the present disclosure relates to a method of producing a stent coated with two cured formulations consisting essentially of the steps of a) providing a stent framework; b) putting the stent framework on a fixture; c) spraying a curable comprising a photoactive compound and a first drug onto the stent framework to provide a curably coated stent; d) covering the curably coated stent with a first glass mask etched with a pattern that selectively allows exposure the curable photosensitive formulation to ultraviolet light; e) curing the curable photosensitive formulation with ultraviolet light thereby forming a substantially cured coated stent; removing the glass mask; f) applying a development solution to the substantially cured coated stent to remove uncured photosensitive formulation; g) drying off excess development solution; and repeating steps b) to g) with a second glass mask etched with a second pattern and a photosensitive formulation comprising a photoactive compound and a second bioactive agent.

The present disclosure also relates to stents coated with multiple cured formulations comprising at least one ultraviolet light cured formulation. Alternatively, the stent comprises a first ultraviolet light cured formulation; and a second ultraviolet light cured formulation; and optionally a third ultraviolet light cured formulation. The ultraviolet light cured formulations each can comprise a bioactive agent and/or polymer. In another embodiment, the stent can comprise a metallic base. The metallic base can be made from a material selected from the group consisting of stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof. Alternatively, each of the ultraviolet cured formulations is in direct contact with the metallic base.

### BRIEF DESCRIPTION OF THE DRAWINGS

FIG. 1a shows a stent framework which has been placed on a fixture.

FIG. 1b shows the application of a curable formulation with a sprayer. The fixture is rotated in this embodiment to promote uniform coverage.

FIG. 1c shows the step of covering the curably coated stent with a glass mask having a pattern which allows selective exposure to ultraviolet light.

FIG. 1d shows the stent which has been covered by a glass mask.

FIG. 1e shows the step of shining ultraviolet light onto the stent covered by the glass mask to cure the formulation is reachable by the ultraviolet light.

FIG. 1f shows removal of the glass mask after curing by ultraviolet light.

FIG. 1g shows application of a development solution.

FIG. 1h shows the drying step.

FIG. 1i shows application of a second ultraviolet curable bioactive agent formulation.

FIG. 1j shows a second exposure to ultraviolet light.

FIG. 1k shows a second application of a development solution.

FIG. 1l shows a second drying step.

### DETAILED DESCRIPTION OF THE INVENTION

The present disclosure generally concerns methods and associated products related to stents coated with multiple formulations. More specifically, selective curing of formulations which may contain bioactive agents and/or polymers after their application onto a provided stent framework results in coated stents having the desired characteristics.

Accordingly, one embodiment of the present disclosure relates to a method of producing a stent coated with n+1 number of formulations comprising the steps of: a) providing a stent framework; b) putting the stent framework on a fixture; c) applying a curable formulation onto the stent framework to provide a curably coated stent; d) covering the curably coated stent with a glass mask etched with a pattern which allows selective exposure of the curable formulation; e) curing the curable formulation thereby forming a substantially cured coated stent; f) removing the glass mask; g) applying a development solution to the substantially cured coated stent to removed uncured formulation; h) drying off excess development solution; and i) repeating steps c) to h) n times; and wherein during step i) a glass mask etched with a different pattern and a different curable formulation are used; and wherein n is greater than or equal to 0.

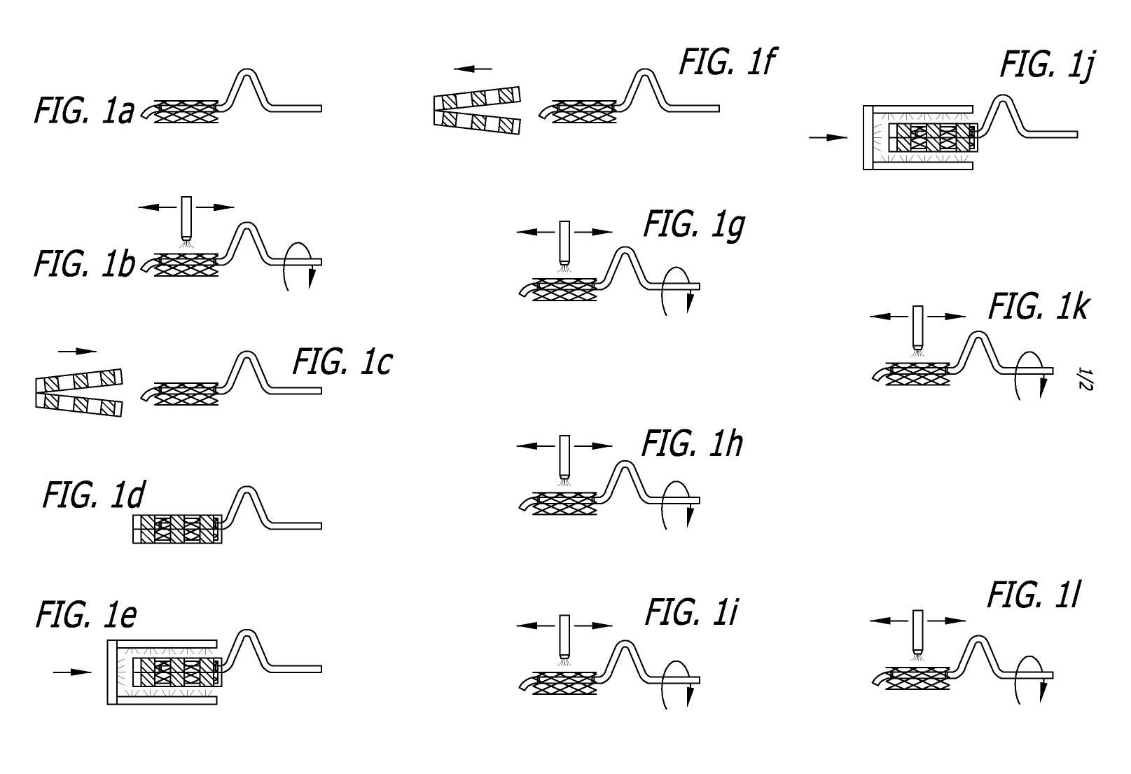

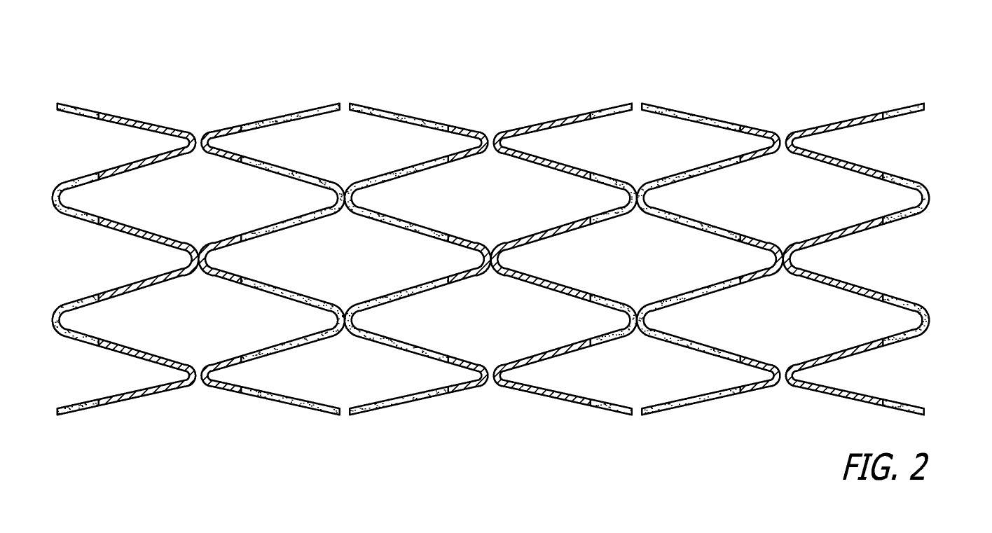

FIG. 1a shows a stent framework which has been placed on a fixture. This fixture preferably allows the stent to rotate and tumble while being coated. The fixture also should allow easy loading and unloading of the stent framework before and after coating. FIG. 1b shows application of a curable formulation with a sprayer. To assist in achieving uniform coverage the fixture can be rotated as shown. Alternatively, application of a curable formulation can be made by other means such as dipping. The provided stent framework can comprise a polymer base or a metallic base. When it is a metallic base it can be made of, but not limited to, stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof. Preferably, the stent framework is made of material that allows strong adherence of the curable formulation, especially after it has been cured.

Before the application of a curable formulation (as shown in FIG. 1b), alternatively, a primer can be coated onto the stent framework. The primer can be selected from, but not limited to, the group consisting of parylene, polyurethane, phenoxy, epoxy, polyimide, polysulfone, and pellathane.

A curable formulation within the scope and meaning of the present invention can be cured by a variety of means including by ultraviolet light. For curing by ultraviolet light, the curable formulation may be a photosensitive formulation comprising a photoactive compound such as a photoinitiator. Photoinitiators are chemicals that form energetic radical species when exposed to UV light. They can be an important ingredient in ultraviolet curable formulations because they allow the polymerization process for curing. Examples of photoinitiators include acylphosphineoxide, phenyl ketone, and hydroxyl ketone.

After application of a curable formulation onto the stent framework (such as shown in FIG. 1b) is completed, a curably coated stent is produced. This stent has curable formulation which has been applied but which has yet been cured.

Next, the curably coated stent is covered with a glass mask etched with a pattern which allows selective exposure of the curable formulation (FIGS. 1c and 1d). Ideally, the glass mask will have been manufactured so that it can be easily mounted on the provided stent framework. The etched pattern on the glass allows a pattern light (such as UV light) through the glass and hence onto the curable formulation applied onto a stent framework. In the embodiment where curing occurs with UV light (FIG. 1e), the parts of the coating exposed to UV light are cured and the parts hidden or covered by the glass mask are not. Therefore, one can achieve selective curing of the applied formulation.

After removing the glass mask (FIG. 1f), a development solution is applied to the cured coated stent (FIG. 1g). This solution can be applied in a variety of ways and includes the method of spraying or dipping. The development solution erodes the parts of the coating which has not been cured (for e.g., because of lack of exposure to UV light). Therefore, after the step of applying a development solution to the cured coated stent, only the coating that has been cured is left behind. The pattern of the remaining coating has been directed by the etched pattern on the glass mask. Excess development solution then can be dried off, for example, with the use of a drying nozzle (FIG. 1h).

Alternatively, in order to have more than one cure formulation on the provided stent framework steps c) to h) above can be repeated. In each repetition however, a glass mask etched with a different pattern and a different curable formulation may be used. In addition, it is within the scope of teachings of the invention however to use a glass mask with the same pattern and same curable formulation in steps c) to h). Steps c) to h) would be repeated once to produce two cured formulations on the stent framework and repeated twice to get three cured formulations on the stent framework and repeated three times to get four cured formulations on the stent framework and so on. When steps c) to h) are repeated, depending on the pattern of the glass etching, the resultant cured multiple formulations may overlap each other or not overlap each other. The resultant cured multiple formulations also may be in direct contact with the initially provided stent framework. When for example, the stent framework is made out of a metal such as stainless steel, some, parts of some, or all of the cured formulations may be in direct contact with the stainless steel. This may be true regardless of what the stent framework is made of within the scope and teaching of the present invention. As an example, the stent framework may be made out of a polymeric base. In one embodiment, steps c) to h) are repeated once, twice or three times.

The development solution could be solvent-based, for example isopropyl or ethyl alcohol. This would remove the uncured coating. The partially coated stent may be then rinsed in non-development solution, such as deionised or distilled water, to remove any remains of the initial development solution and prepare the stent for the next coating layer(s).

In another embodiment, the curable formulation further comprises a bioactive agent. As an example. the bioactive agent may be an antirestenotic drug or selected from the group consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof.

The bioactive agent can be an agent against one or more conditions including coronary restenosis, cardiovascular restenosis, angiographic restenosis, arteriosclerosis, hyperplasia, and other diseases and conditions. For example, the bioactive agent may be selected to inhibit or prevent vascular restenosis, a condition corresponding to a narrowing or constriction of the diameter of the bodily lumen where the stent is placed. The bioactive agent can also be a cocktail or combination of therapeutic drugs. A number of pharmaceutical drugs have the potential to be used in bioactive agent-polymer coatings. For example, an antirestenotic agent such as rapamycin prevents or reduces the recurrence of narrowing and blockage of the bodily vessel. An antisense agent works at the genetic level to interrupt the process by which disease-causing proteins are produced. An antineoplastic agent is typically used to prevent, kill, or block the growth and spread of cancer cells in the vicinity of the stent. An antiproliferative agent may prevent or stop targeted cells or cell types from growing. An antithrombogenic agent actively retards blood clot formation. An anticoagulant often delays or prevent blood coagulation with anticoagulant therapy, using compounds such as heparin and coumarins. An antiplatelet agent may be used to act upon blood platelets, inhibiting their function in blood coagulation. An antibiotic is frequently employed to kill or inhibit the growth of microorganisms and to combat disease and infection. An anti-inflammatory agent such as dexamethasone can be used to counteract or reduce inflammation in the vicinity of the stent. At times, a steroid may be used to reduce scar tissue in proximity to an implanted stent. A gene therapy agent may be capable of changing the expression of a person's genes to treat, cure or ultimately prevent disease.

Within the scope and teachings of the present invention a bioactive agent is any therapeutic substance that provides treatment of disease and/or conditions. An organic drug is any small-molecule therapeutic material. A pharmaceutical compound is any compound that provides a therapeutic effect. A recombinant DNA product or recombinant RNA product includes altered DNA or RNA genetic material. Bioactive agents of pharmaceutical value may also include collagen and other proteins, saccharides, and their derivatives. The molecular weight of the bioactive agent typically range from 200 to 600,000 Daltons and above.

Alternatively, the curable formulation may further comprise a polymer. The polymer can be, for example, selected from the group consisting of polyurethane, silicone, polyolefin, polyisobutylene, ethylene-alphaolefin copolymer, acrylic polymer and copolymer, ethylene-co-vinylacetate, polybutylmethacrylate, vinyl halide polymer and copolymer, polyvinyl chloride, polyvinyl ether, polyvinyl methyl ether, polyvinylidene halide, polyvinylidene fluoride, polyvinylidene chloride, polyacrylonitrile, polyvinyl ketone, polyvinyl aromatic, polystyrene, polyvinyl ester, polyvinyl acetate, ethylene-methyl methacrylate copolymer, acrylonitrile-styrene copolymer, ABS resin, ethylene-vinyl acetate copolymer, polyamide, alkyd resin, polycarbonate, polyoxymethylene, polyimide, polyether, epoxy resin, polyurethane, rayon, rayon-triacetate, cellulose, cellulose acetate, cellulose butyrate, cellulose acetate butyrate; cellophane, cellulose nitrate, cellulose propionate, cellulose ether, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylate, poly(trimethylene carbonate), poly(iminocarbonate), copoly(ether-esters), polyalkylene oxalate, polyphosphazene, fibrin, fibrinogen, cellulose, starch, collagen and hyaluronic acid, and combinations thereof.

In another embodiment, the curable formulation may further comprise a bioactive agent and a polymer. Thus, curable formulation could contain a “bioactive agent-polymer.” The above listed bioactive agents and polymers can be used in the combination to form a bioactive agent-polymer in the cured formulation. Bioactive agent-polymer coating may include and elute multiple bioactive agents. Also, the bioactive agent-polymer coating can be tailored to control the elution of one or more bioactive agents primarily by diffusion processes. In some cases, a portion of the polymeric coating is absorbed into the body to release bioactive agents from within the coating

Bioactive agent-polymer coating can contain a polymeric blend wherein the polymeric blend comprises a fractional part of the styrenic block copolymer based on a predetermined elution rate of the bioactive agent. Modification of the polymeric blend allows, for example, rapid delivery of a pharmacologically active drug or bioactive agent within twenty-four hours of a surgery, with a slower, steady delivery of a second bioactive agent over the next three to six months.

Another embodiment of the present disclosure relates to a method of producing a stent coated with two cured formulations consisting essentially of the steps of: a) providing a stent framework; b) putting the stent framework on a fixture; c) spraying a curable photosensitive formulation comprising a photoactive compound and a first drug onto the stent framework to provide a curably coated stent; d) covering the curably coated stent with a first glass mask etched with a pattern that selectively allows exposure of the curable photosensitive formulation to ultraviolet light; e) curing the curable photosensitive formulation with ultraviolet light thereby forming a substantially cured coated stent; f) removing the glass mask; g) applying a development solution to the substantially cured coated stent to remove uncured photosentive formulation; h) drying off excess development solution; and i) repeating steps c) to h) with a second glass mask etched with a second pattern and a second curable photosensitive formulation comprising a photoactive compound and a second bioactive agent.

Another aspect of the present disclosure relates to a stent coated with multiple cured formulations comprising at least one ultraviolet light cured formulation. In another embodiment, the stent coated with multiple cured formulations comprises a first ultraviolet light cured formulation, a second ultraviolet light cured formulation; and optionally a third ultraviolet light cured formulation. In these embodiments the ultraviolet light cured formulation can comprise a bioactive agent and/or a polymer. In another embodiment, the stent can comprise a metallic base and metallic base is selected from the group consisting of stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof. Alternatively, each of the ultraviolet cured formulation is in direct contact with the metallic base. Each ultraviolet cured formulation may be uniformly coated. However, the pattern of each coating may also be non-uniform, for example, in the form of stripes or dots. This is true for the methods disclosed herein as well and the stents produced by the presently disclosed methods.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of producing a stent coated with n+1 number of cured formulations comprising the steps of:
a) providing a stent framework;
b) putting said stent framework on a fixture;
c) applying a curable formulation onto said stent framework to provide a curably coated stent;
d) covering said curably coated stent with a glass mask etched with a pattern which allows selective exposure of said curable formulation;
e) curing said curable formulation thereby forming a substantially cured coated stent;
f) removing said glass mask;
g) applying a development solution to said substantially cured coated stent to remove uncured formulation;
h) drying off excess development solution; and
i) repeating steps c) to h) n times; and
wherein during said step i) a glass mask etched with a different pattern and a different curable formulation are used; and
wherein n is greater than or equal to 0.

2. The method of claim 1, wherein said curable formulation is a photosensitive curable formulation comprising a photoactive compound.

3. The method of claim 2, wherein said photosensitive curable formulation is sensitive to ultraviolet light.

4. The method of claim 1, wherein said curing is performed with ultraviolet light.

5. The method of claim 1, wherein said curable formulation further comprises a bioactive agent.

6. The method of claim 1, wherein said curable formulation further comprises a polymer.

7. The method of claim 1, wherein said curable formulation further comprises a bioactive agent and a polymer.

8. The method of claim 5, wherein said bioactive agent comprises an antirestenotic drug.

9. The method of claim 5, wherein said bioactive agent is selected from a group consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof.

10. The method of claim 6, wherein said polymer is selected from the group consisting of polyurethane, silicone, polyolefin, polyisobutylene, ethylene-alphaolefin copolymer, acrylic polymer and copolymer, ethylene-co-vinylacetate, polybutylmethacrylate, vinyl halide polymer and copolymer, polyvinyl chloride, polyvinyl ether, polyvinyl methyl ether, polyvinylidene halide, polyvinylidene fluoride, polyvinylidene chloride, polyacrylonitrile, polyvinyl ketone, polyvinyl aromatic, polystyrene, polyvinyl ester, polyvinyl acetate, ethylene-methyl methacrylate copolymer, acrylonitrile-styrene copolymer, ABS resin, ethylene-vinyl acetate copolymer, polyamide, alkyd resin, polycarbonate, polyoxymethylene, polyimide, polyether, epoxy resin, polyurethane, rayon, rayon-triacetate, cellulose, cellulose acetate, cellulose butyrate, cellulose acetate butyrate; cellophane, cellulose nitrate, cellulose propionate, cellulose ether, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylate, poly(trimethylene carbonate), poly(iminocarbonate), copoly(ether-esters), polyalkylene oxalate, polyphosphazene, fibrin, fibrinogen, cellulose, starch, collagen and hyaluronic acid, and combinations thereof.

11. The method of claim 7, wherein said bioactive agent is selected from the consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof;
and said polymer is selected from the group consisting of polyurethane, silicone, polyolefin, polyisobutylene, ethylene-alphaolefin copolymer, acrylic polymer and copolymer, ethylene-co-vinylacetate, polybutylmethacrylate, vinyl halide polymer and copolymer, polyvinyl chloride, polyvinyl ether, polyvinyl methyl ether, polyvinylidene halide, polyvinylidene fluoride, polyvinylidene chloride, polyacrylonitrile, polyvinyl ketone, polyvinyl aromatic, polystyrene, polyvinyl ester, polyvinyl acetate, ethylene-methyl methacrylate copolymer, acrylonitrile-styrene copolymer, ABS resin, ethylene-vinyl acetate copolymer, polyamide, alkyd resin, polycarbonate, polyoxymethylene, polyimide, polyether, epoxy resin, polyurethane, rayon, rayon-triacetate, cellulose, cellulose acetate, cellulose butyrate, cellulose acetate butyrate; cellophane, cellulose nitrate, cellulose propionate, cellulose ether, carboxymethyl cellulose, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylate, poly(trimethylene carbonate), poly(iminocarbonate), copoly(ether-esters), polyalkylene oxalate, polyphosphazene, fibrin, fibrinogen, cellulose, starch, collagen and hyaluronic acid, and combinations thereof.

12. The method of claim 1, wherein n is 0, 1, 2, or 3.

13. The method of claim 1, wherein said stent framework comprises a metallic base.

14. The method of claim 13, wherein said metallic base is selected from the group consisting of stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof.

15. The method of claim 1, wherein said stent framework comprises a polymeric base.

16. The method of claim 1, further comprising coating said stent framework with a primer prior to step b).

17. The method of claim 16, wherein said primer is selected from the group consisting of parylene, polyurethane, phenoxy, epoxy, polyimide, polysulfone, and pellathane.

18. The method of claim 2, wherein said photoactive compound is a photoinitiator.

19. The method of claim 1, wherein said applying in steps c) and g) is spraying.

20. A method of producing a stent coated with two cured formulations consisting essentially of the steps of:
a) providing a stent framework
b) putting said stent framework on a fixture;
c) spraying a curable photosensitive formulation comprising a photoactive compound and a first drug onto said stent framework to provide a curably coated stent;
d) covering said curably coated stent with a first glass mask etched with a pattern that selectively allows exposure of said curable photosensitive formulation to ultraviolet light;
e) curing said curable photosensitive formulation with ultraviolet light thereby forming a substantially cured coated stent;
f) removing said glass mask;
g) applying a development solution to said substantially cured coated stent to remove uncured photosentive formulation;
h) drying off excess development solution; and
i) repeating steps c) to h) with a second glass mask etched with a second pattern and a second curable photosensitive formulation comprising a photoactive compound and a second bioactive agent.

21. A stent coated with multiple cured formulations comprising:
at least one ultraviolet light cured formulation.

22. The stent of claim 21, wherein said stent comprises:
a first ultraviolet light cured formulation; and
a second ultraviolet light cured formulation; and
optionally a third ultraviolet light cured formulation.

23. The stent of claim 21, wherein said ultraviolet light cured formulation comprises a bioactive agent and/or a polymer.

24. The stent of claim 21, wherein said stent comprises a metallic base.

25. The stent of claim 24, wherein said metallic base is selected from the group consisting of stainless steel, nitinol, tantalum, a nonmagnetic nickel-cobalt-chromium-molybdenum [MP35N] alloy, platinum, titanium, a suitable biocompatible alloy, a suitable biocompatible material, and a combination thereof.

26. The stent of claim 24, wherein each of said ultraviolet cured formulation is in direct contact with said metallic base.
